(19) United States
(12) Patent Application Publication
(10) Pub. No.: US 2005/0256562 A1
(43) Pub. Date: Nov. 17, 2005

(54) Stent delivery handle and assembly formed therewith

(75) Inventors: Claude Clerc, Marlborough, MA (US); Roy H. Sullivan, Millville, MA (US); John B. Golden, Norton, MA (US); Gary Jordan, Litchfield, NH (US)

Correspondence Address: HOFFMANN & BARON, LLP, 6900 JERICHO TURNPIKE, SYOSSET, NY, 11791, US

(73) Assignee: Boston Scientific Scimed, Inc.

(21) Appl. No.: 11/125,722

(22) Filed: May 10, 2005

Related U.S. Application Data
(60) Provisional application No. 60/571,140, filed on May 14, 2004.

(51) Int. Cl. A61F 2/06

(52) U.S. Cl. 623/1.11

(57) ABSTRACT

A delivery handle and assembly are provided which allow for deployment and reconstrainment of a stent. The delivery assembly may include a catheter having a lumen extending therethrough; and, a housing having an aperture, the catheter extending through the aperture. A driver is movably coupled to the housing such that the driver can selectively move in different first and second directions relative to the housing. The driver is disposed to engage the catheter such that movement of the driver in the first direction causes a distal end of the catheter to move distally, and that movement of the driver in the second direction causes the distal end of the catheter to move proximally.

### CROSS-REFERENCE TO RELATED APPLICATION

This application claims priority of U.S. Provisional Patent Application No. 60/571,140, filed May 14, 2004, the entire contents of which are incorporated by reference herein.

### BACKGROUND OF THE INVENTION

This invention relates to stent delivery devices, and, more particularly, to handle mechanisms for stent delivery.

Catheter systems for deploying stents are well known in the art. Various catheter systems are known which rely on a guidewire for navigation, such as over-the-wire systems, rapid exchange systems, and fixed wire systems. Certain stent applications do not require navigation of a catheter through a tortuous pathway and, as such, do not require a guidewire steering mechanism.

Common catheter systems require manual manipulation of various coaxially disposed elements, such as catheters, sheaths, pushers, guidewires, and so forth, to allow for deployment of a stent or other treatment device at a desired location. Handles have been developed in the prior art to allow for trigger-actuated deployment, such as with the “pistol grip” actuator disclosed in U.S. Published Patent Application No. 2002/0183826 A1, published on Dec. 5, 2002 to Dorn, et al. These devices, however, are “one-way” devices, which allow for deployment of a stent, but not reconstrainment. Thus, the re-positioning of a partially deployed stent with the “pistol grip” device may be difficult, particularly where the stent has been fairly deployed and is engaging the walls of the surrounding bodily passageway.

### SUMMARY OF THE INVENTION

A delivery handle and assembly formed therewith is provided herein which allows for deployment and reconstrainment of a stent. In one broad aspect of the subject invention, a stent delivery assembly is provided which includes a catheter having a lumen extending therethrough; and, a housing having an aperture, the catheter extending through the aperture. A driver is movably coupled to the housing such that the driver can selectively move in a first direction relative to the housing, and in a second direction, different from the first direction, relative to the housing. Further, the driver is disposed to engage the catheter such that movement of the driver in the first direction causes a distal end of the catheter to move distally, and that movement of the driver in the second direction causes the distal end of the catheter to move proximally.

Advantageously, with the subject invention, proximal and distal movement of the catheter distal end is achievable to selectively deploy and reconstrain a stent. In this manner, accurate placement of the stent at a desired location may be achieved.

It must be noted that the subject invention is useable to deploy devices other than stents. For example, the subject invention may be used to deploy stone (e.g., kidney stone) retrieval baskets, injection needles (e.g., sclerotherapy needles, needles for injectable endoscopic therapy, and transbronchial aspiration needles), and inflatable balloon products. The subject invention is particularly well-suited for use with stents, but can be used in these other applications.

In one variation, the catheter may be provided as a fixed guidewire system which is not well suited for navigation through a tortuous pathway. Alternatively, the housing may be provided with a rear port, and the catheter may be slitted in proximity to its proximal end, thereby allowing a guidewire to be thread through the port and the slit of the catheter to allow for an over-the-wire or rapid exchange configuration. Thus, the guidewire may be initially navigated through a bodily passageway with a steering mechanism, as known in the art, with subsequent mounting of the housing onto the guidewire, after removal of the steering mechanism.

In a preferred embodiment, the driver is wheel-shaped and rotatable in clockwise and counter-clockwise directions. Also, the housing is formed to have a handle for engagement by an operator. It is further preferred that the driver and catheter have shape-mating configurations which allow for enhanced engagement. For example, the driver may be formed as a gear, and the catheter may be provided with a corrugated portion, such that rotational movement of the driver results in linear translation of the catheter in a manner similar to a rack and pinion arrangement.

These and other features of the invention will be better understood through a study of the following detailed description and accompanying drawings.

### BRIEF DESCRIPTION OF THE DRAWINGS

FIG. 1 is a perspective view of an embodiment of the subject invention;

FIG. 2 is a cut-away view of the embodiment of FIG. 1;

FIG. 3 shows a portion of the catheter configured for shape-mating engagement with a driver of the subject invention;

FIG. 4 is an enlarged view of Section 4 in FIG. 3;

FIG. 5 is a cut-away view of an embodiment of the subject invention, wherein the idler wheel and driver are formed for shape-mating engagement with a portion of the catheter;

FIG. 6 is a perspective view of an embodiment of the subject invention useable with a generally cylindrical catheter;

FIG. 7 is a cut-away view of the embodiment of FIG. 6;

FIGS. 8-9 show different handle configurations useable with the subject invention;

FIGS. 10 and 11 depict a locking mechanism useable with the subject invention;

FIG. 12 is a schematic of a variation of the subject invention, wherein a rear port is provided in the housing to accept a guidewire;

FIGS. 13 and 14 depict a process of using the subject invention with a fixed guidewire catheter configuration; and,

FIGS. 15 and 16 depict a process of using the subject invention with an over-the-wire catheter configuration.

### DETAILED DESCRIPTION OF THE INVENTION

A device is provided herein, which is designated with reference numeral 10, for deploying a stent, or other device described above, in a bodily passageway. Deployment can be achieved in the coronary or peripheral vasculature, pulmonary tract, esophagus, trachea, colon, biliary tract, urinary tract, prostate or brain. Reference to bodily passageway may be to any one of these passages or elsewhere in the body.

It should be noted that references herein to the term “distal” are to a direction away from an operator of the subject invention, while references to the term “proximal” are to a direction towards the operator of the subject invention.

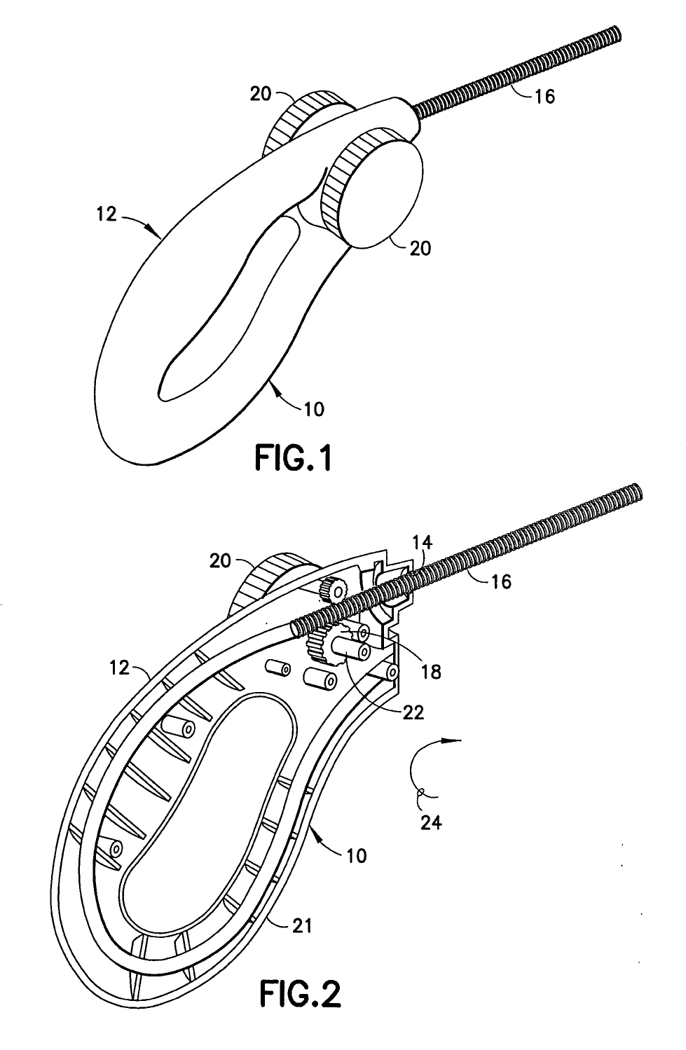

As shown in FIGS. 1 and 2, the device 10 includes a housing 12 formed with an aperture 14 through which a portion of a catheter 16 extends through. A driver 18 is movably coupled to the housing 12 such that the driver 18 may move in a first direction relative to the housing 12 and in a second direction, different from the first direction, preferably, opposite the first direction, relative to the housing 12. In a preferred embodiment, the driver 18 is wheel-shaped and coupled to allow for clockwise and counter-clockwise rotation relative to the housing 12. It is preferred that the driver 18 be wholly enclosed within the housing 12 and that at least one external knob 20 be provided which is coupled to the driver 18 through the wall of the housing 12 such that movement of the external knob 20 results in corresponding movement of the driver 18. Again, with the preferred embodiment, the external knob 20 is generally wheel-shaped, with clockwise rotation of the external knob 20 resulting in clockwise rotation of the driver 18 and counter-clockwise rotation of the external knob 20 resulting in counter-clockwise rotation of the driver 18. To facilitate left- and right-handed operators, two of the external knobs 20 may be provided on opposite sides of the housing 12 which are both fixed to the driver 18 as indicated above. Accordingly, the driver 18 and the two external knobs 20 may move in concert. By way of non-limiting example, the external knobs 20 may be coupled to the driver 18 by being mounted to pins 22 rigidly extending from the driver 18.

The aperture 14 is preferably located to axially align the catheter 16 to engage the driver 18. It is preferred that the driver 18 tangentially engage the catheter 16. With rotation of the driver 18, forces will be imparted to the catheter 16 to cause linear translation thereof. Thus, clockwise rotation of the driver 18, as represented by arrow 24, will result in movement of the catheter 16 in a distal direction. Conversely, rotation of the driver 18 in the opposite, counter-clockwise direction, will result in the catheter 16 moving in a proximal direction. To ensure proper engagement between the catheter 16 and the driver 18, a follower, idler wheel 26 may be provided. Preferably, the idler wheel 26 is spaced from the driver 18 at the point at which engagement with the catheter 16 is desired. In this manner, a nip is defined through which the catheter 16 extends. The idler wheel 26 is preferably freely rotatable in both directions.

The housing 12 may include a channel 21 to accommodate the catheter 16. Proximal movement of the catheter 16 may be limited by the length of the channel 21. Accordingly, a length may be chosen to prevent unnecessary proximal movement of the catheter, yet sufficient proximal movement to permit deployment of the stent as described below.

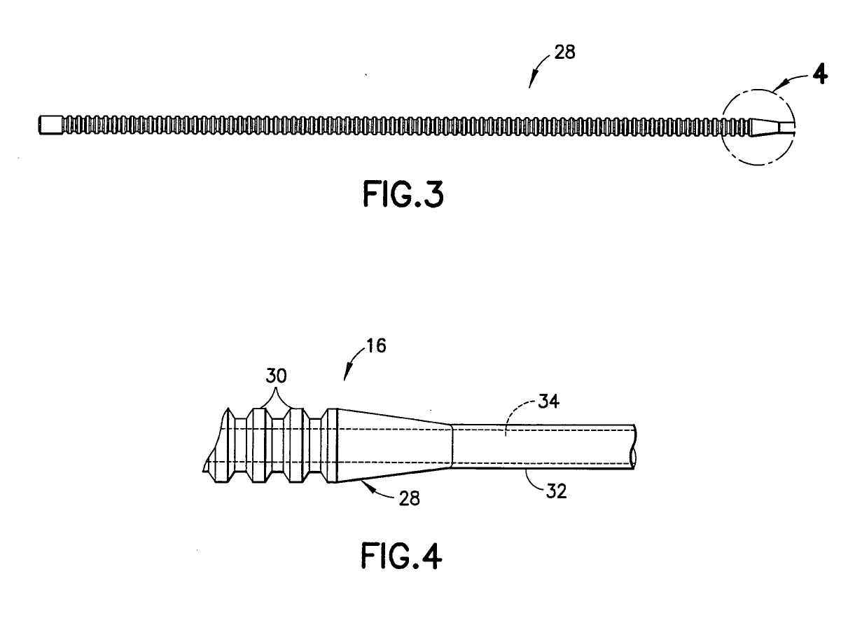

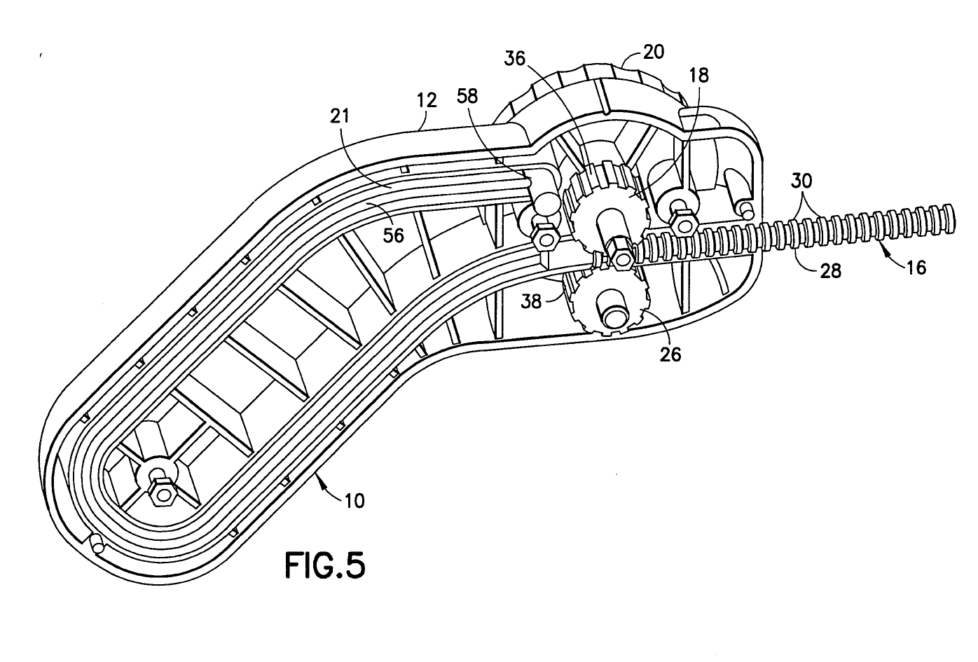

It is preferred that the driver 18 and at least a portion of the catheter 16 be formed with shape-mating configurations to enhance inter-engagement therebetween. With shape-mating inter-engagement, mechanical interaction is provided in addition to frictional engagement. As shown in FIGS. 3-5, a section 28 of the catheter 16 may be formed with corrugations 30. The section 28 may be unitarily formed with a remainder section 32 of the catheter 16 being generally smooth and cylindrical, thus adaptable for insertion into a bodily passageway. The sections 28 and 32 may be unitarily formed or formed separately and joined together. With the sections 28 and 32 being separately formed, different materials can be used which provide different characteristics. For example, a relatively stiff polymer (e.g., nylon 12; thermoplastic polyester elastomer) may form the section 32 while a more flexible polymer (e.g., nylon; polyether-block co-polyamide polymer) may be used to form the section 28. With flexibility, the section 28 provides strain relief to the catheter 16 at the aperture 14. A continuous lumen 34 is defined between the two sections 28 and 32 which extends throughout the full length of the catheter 16.

With the section 28 being corrugated, the driver 18 may be gear-shaped with radially-spaced apart teeth 34 extending from its periphery formed for meshing engagement with the corrugations 30. Likewise, teeth 36 may be provided at radially-spaced apart locations about the periphery of the idler wheel 26. Meshing engagement of the teeth 34 with the corrugations 30 facilitates distal and proximal translation of the catheter 16. Advantageously, the shape-mating engagement eliminates the need to generate high frictional forces at the nip between the driver 18 and the idler wheel 26.

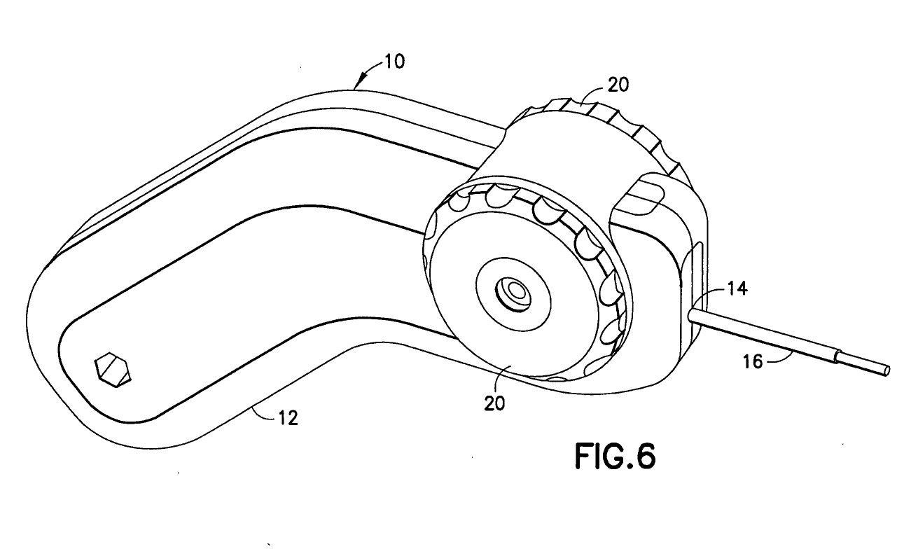

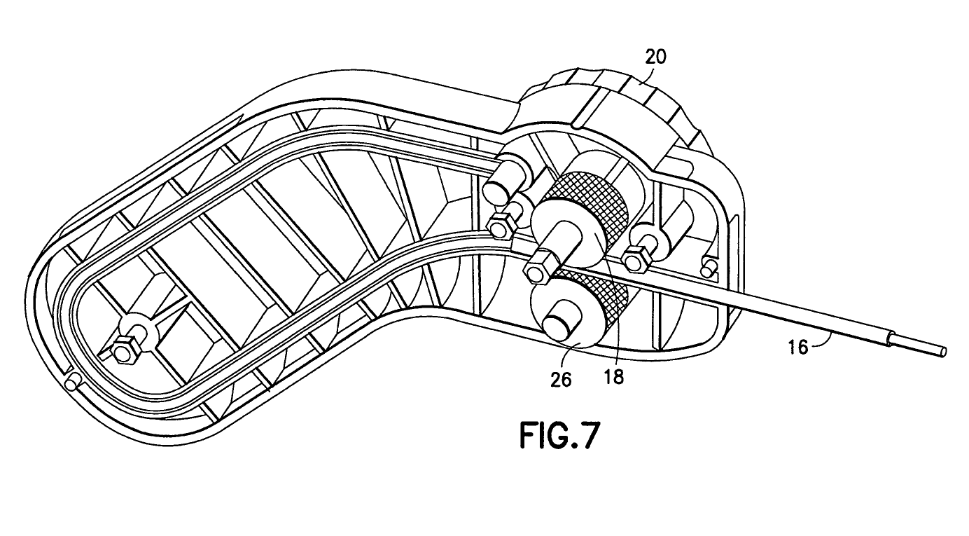

In an alternate configuration, wherein the catheter 16 is formed with a smooth cylindrical shape throughout, the driver 18 and/or the idler wheel 26 may be formed with a knurled or textured surface to enhance frictional engagement with the catheter 16, as shown in FIGS. 6 and 7. It is preferred that the nip between the driver 18 and the idler wheel 26 be defined and positioned to ensure sufficient frictional force will be generated to act on the catheter 16 in causing translation thereof. Thus, it is preferred that the nip be slightly smaller than the outer diameter of the catheter 16. The durometer and other characteristics of the material comprising the catheter 16 should be considered in sizing the nip between the driver 18 and the idler wheel 26.

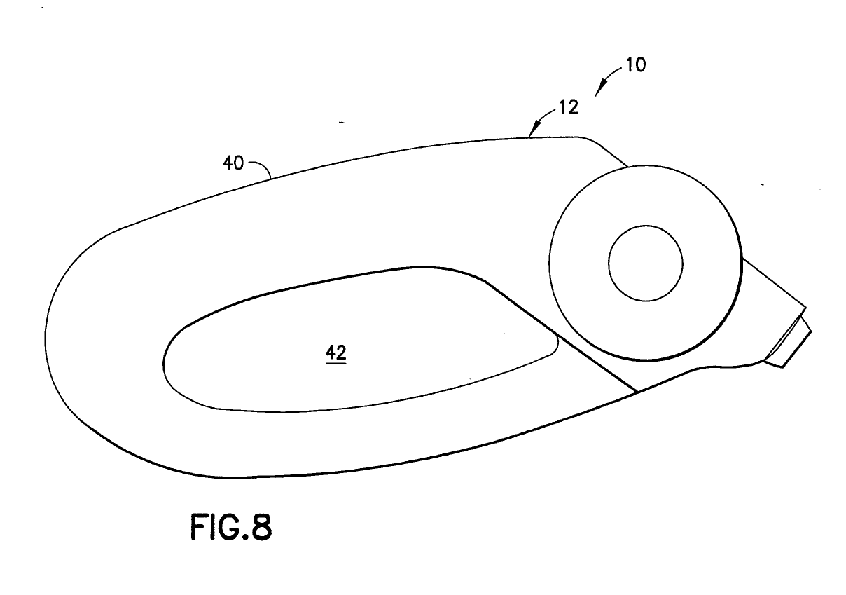

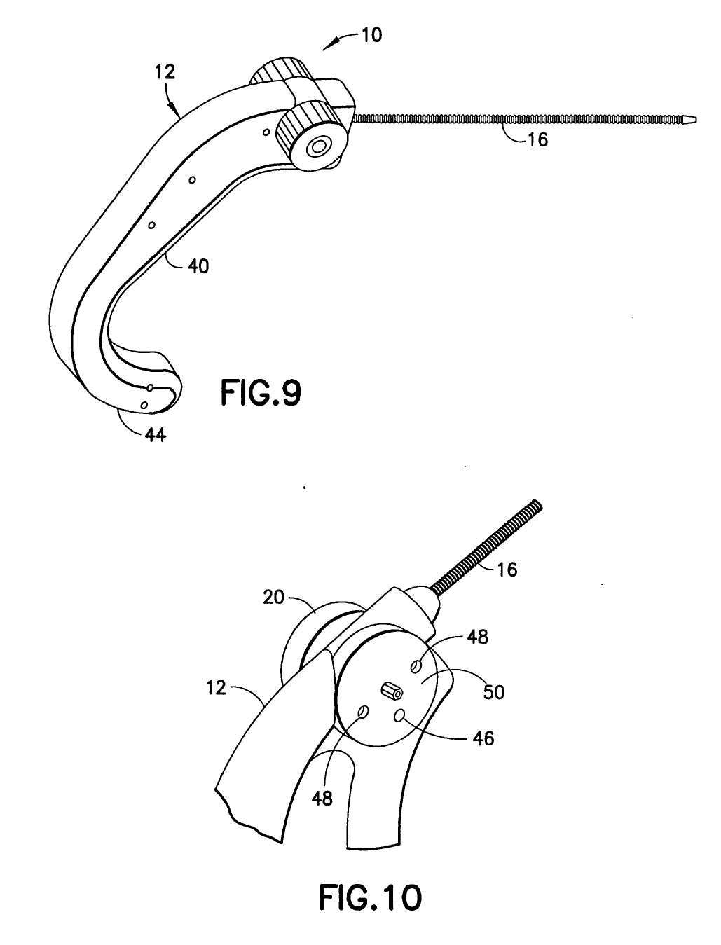

It is preferred that the housing 12 be formed to include a handle section 40 which is sized and shaped to be comfortably gripped by an operator of the device 10, thereby reducing operator fatigue. In a preferred embodiment, as shown in FIGS. 1, 2 and 8, the handle section 40 completely encircles a finger receiving aperture 42. Other configurations of the handle section 40 are possible. With reference to FIGS. 5 and 6, the handle section 40 may be shaped similarly to a pistol grip, while with reference to FIG. 9, the handle section 40 may terminate in a hooked shaped end 44.

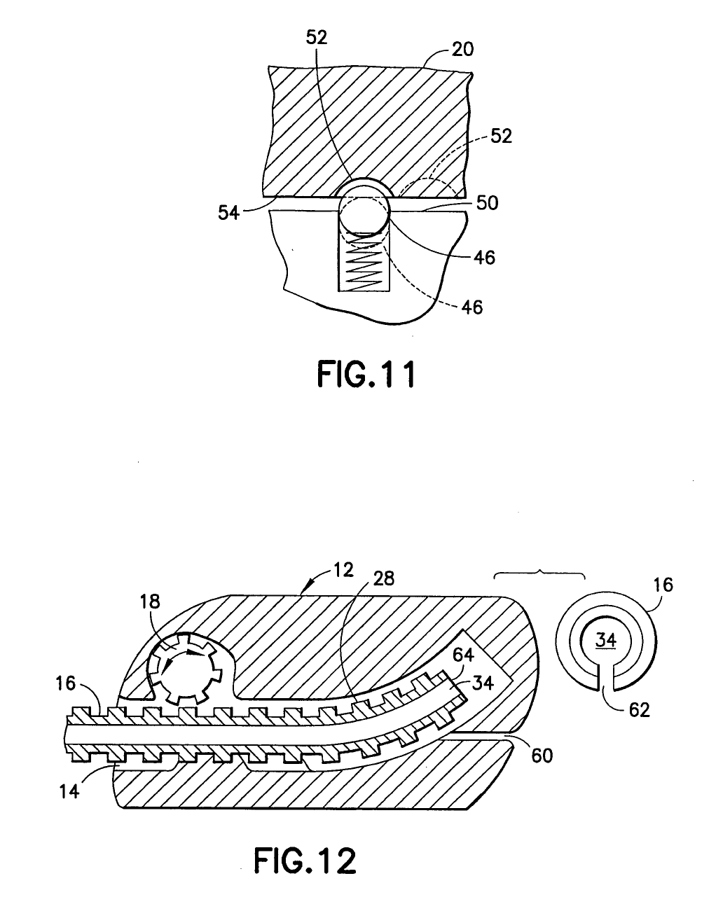

As will be appreciated by those skilled in the art, free rotation of the driver 18 is not desired. Frictional engagement between the driver 18, the external knobs 20, the pins 22 and the housing 12 may act to restrict free rotation of the driver 18. Of course, excessive restriction is also not desired. Preferably, a locking arrangement is provided wherein the driver 18 may be fixed at various radial positions during use. With reference to FIGS. 10 and 11, an exemplary locking mechanism is depicted. Herein, one or more spring-biased balls 46 are disposed within the housing 12 so as to partially extend therefrom towards the external knobs 20. Openings 48 allow for partial passage of the balls 46, but not for complete passage thereof. The balls 46 are spaced apart on faces 50 that are located to be opposite the external knobs 20 during use. Ball receiving pockets 52 are formed on inner faces of the external knobs 20, as shown in FIG. 11. The ball receiving pockets 52 are shaped and positioned to receive the balls 46 in outward-most extending positions when aligned with the balls 46. Upon rotation of the external knobs 20, intermediate sections 54 defined on the external knobs 20 between the ball receiving pockets 52 engage and press down the balls 46, thus, freeing the balls 46 from the ball receiving pockets 52. Upon sufficient rotation of the external knobs 20, the balls 46 spring into the next occurring ball receiving pockets 52. With engagement between the balls 46 and the ball receiving pockets 52, both tactile and audible clicks can be formed to indicate positional adjustment to an operator. Any combination of the number of the balls 46 and the ball receiving pockets 52 can be utilized to allow for greater and less frequent position fixing. With the balls 46 being received within the ball receiving pockets 52, rotational movement of the external knobs 20 is limited, and, thus, the driver 18 is also positionally fixed. A threshold force is required to disengage the balls 46 from the ball receiving pockets 52 and cause positional adjustment of the driver 18. As will be appreciated by those skilled in the art, pressure generated by the spring-biasing force acting against the balls 46, also acts against the external knobs 20 with there being restriction against free-unhindered movement of the external knobs 20, and, thus, restriction against free, unhindered movement of the driver 18.

The device 10 can be used with various catheter configurations, including over-the-wire, rapid exchange, and fixed guidewire configurations. With a fixed guidewire or a rapid exchange configuration, a core element, such as a pusher 56, extending through the lumen 34 of the catheter 16 may be fixed to the housing 12. With reference to FIG. 5, proximal end 58 of the pusher 56 is fixed to a portion of the housing 12. To allow for the over-the-wire configuration, and with reference to FIG. 12, a rear port 60 may be provided formed to allow passage therethrough of a guidewire. In addition, a portion of the catheter 16 may be provided with a slit 62 through which a guidewire may enter the lumen 34. Preferably, the catheter 16 includes the slit 62 only over a limited axial length in proximity to a proximal end 64 of the catheter. Although not shown, a sleeve may extend outwardly from the housing 12 about the catheter 16 and beyond the slit 62 to ensure that no components contained within the lumen 34 are inadvertently released therefrom.

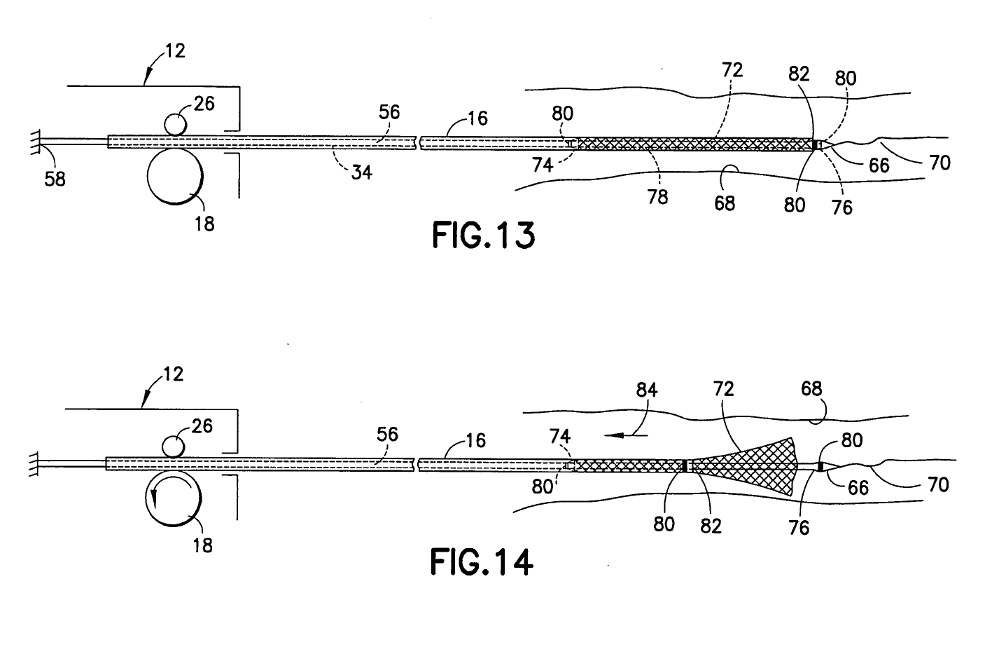

With reference to FIGS. 13 and 14, for illustrative purposes, deployment of a stent is shown using a fixed guidewire type configuration. In particular, the pusher 56 extends through the lumen 34 with the proximal end 58 of the pusher 56 being secured to a portion of the housing 12. The pusher 56 has a distal tip 66 formed for insertion into a bodily passageway 68. A fixed guidewire 70 may extend from the tip 66 to aid in navigation of the assembly. The fixed guidewire 70 may also extend from the housing 12 and through the distal tip 66 (not shown). A stent 72 is collapsed within the lumen 34 of the catheter 16 during insertion. The stent 72 is distensible to a diameter greater than the lumen 34 and may be of any known configuration, including being of the self-expanding type and of the balloon-expandable type. For illustrative purposes, the stent 72 is shown as being of a self-expanding type. The catheter 16 ensheaths the stent 72 until it is ready for deployment. To maintain the stent 72 in a fixed axial position relative to the catheter 16, proximal and distal ferrule-shaped stent retaining members 74, 76 are provided on the pusher 56 which define a stent receiving recess 78 therebetween. Radiopaque markers 80 may be provided adjacent to the stent retaining members 74, 76 to provide indications of the location of the stent 72 during deployment. During initial positioning of the assembly in the bodily passageway 68, distal end 82 of the catheter 16 is located distally of the stent 72. It is preferred that a radiopaque marker 80 also be provided adjacent to the distal end 82.

For deployment, the assembled catheter 16, pusher 56, and stent 72 are inserted into the bodily passageway 68. Using known fluoroscopy techniques, the stent 72 is positioned at a desired location by locating the radiopaque markers 80 about the location. Once positioned, and with reference to FIG. 14, the catheter 16 may be retracted proximally relative to the stent 72 by moving the driver 18 (as shown in FIG. 14, the driver 18 is driven counter-clockwise). With relative proximal movement of the distal end 82 of the catheter 16 in the direction represented by the arrow 84, the stent 72 is caused to be incrementally exposed. As shown in FIG. 14, with a self-expanding type of stent, the stent 72 flares upon exposure in expanding. During deployment, the radiopaque marker 80 adjacent to the distal end 82 of the catheter 16 provides an indication relative to the radiopaque markers 80 located adjacent to the stent retaining members 74, 76 as to the length of the stent 72 which has been exposed. Sufficient relative proximal movement of the distal end 82 of the catheter 16 results in full exposure of the stent 72 in causing deployment thereof. If repositioning of the stent 72 is required during deployment, the driver 18 can be forced into the opposite direction to cause distal movement of the distal end 82 relative to the stent, thereby causing at least partial reconstrainment of the stent 72. The stent 72 can be sufficiently reconstrained to avoid excessive engagement between the stent 72 and the walls of the bodily passageway 68 in allowing for repositioning. Once correctly re-positioned, the driver 18 can once again cause relative proximal movement of the distal end 82.

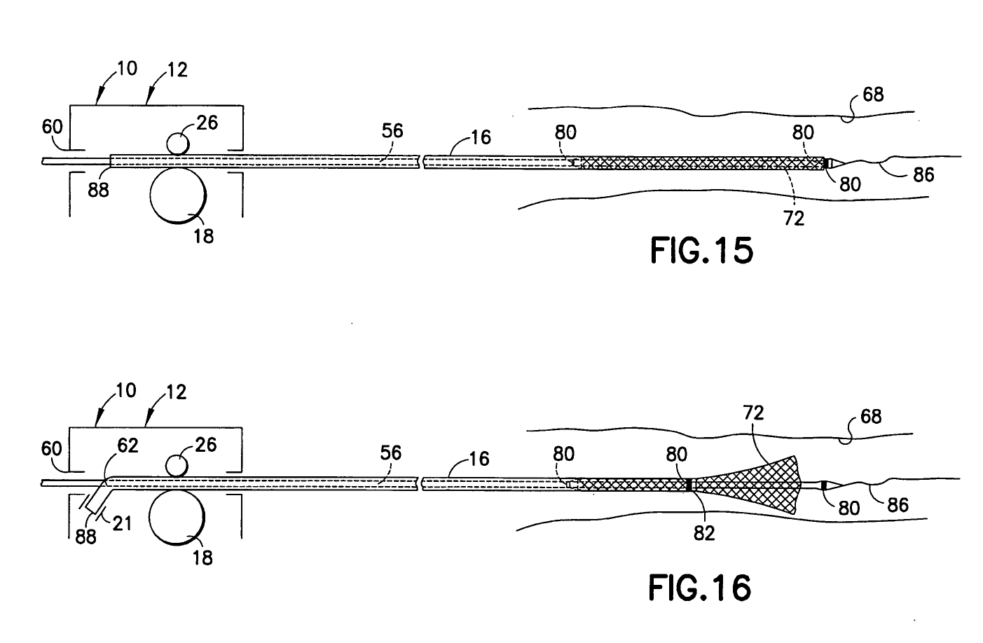

With reference to FIGS. 15 and 16, for illustrative purposes, a method of using the subject invention with over-the-wire (depicted) and rapid exchange (not depicted) catheter configurations is illustrated. The configuration of the catheter 16 and the pusher 56 are generally the same as above. Here, however, the pusher 56 includes a lumen extending at least along a part of the length thereof through which a guidewire 86 extends. The length of the lumen through the pusher 56 will depend on the catheter configuration (over-the-wire or rapid exchange). For deployment, the guidewire 86 may be navigated into the bodily passageway 68 using known steering mechanisms. Once positioned, the steering mechanism (not shown) may be removed, with the guidewire 86 being maintained in place. The device 10 is then thread onto a proximal end of the guidewire 86 with the pusher 56 and the catheter 16 being likewise threaded thereonto, either in an over-the-wire configuration, as shown in the figures, or alternatively in a rapid exchange fashion. The guidewire may extend from a proximal end 88 of the catheter 16 and through the rear port 60, as shown in FIG. 15. Alternatively, as shown in FIG. 16, and as described above, the guidewire 86 may extend through the slit 62 formed in the catheter 16 and through the rear port 60. With this configuration, the catheter 16 may have its proximal end 88 disposed in the channel 21 formed in the housing 12 to limit proximal movement thereof.

Once prepared, the catheter 16, pusher 56, and stent 72 assembly can be slid over the guidewire 86 and positioned using the radiopaque markers 80 and fluoroscopy techniques, as described above. Once positioned, the guidewire 86 is held in a fixed position, and the distal end 82 of the catheter 16 is caused to move proximally relative to stent 72 by the driver 18 in the same manner as described above. The stent 72 may be reconstrained as needed to allow for proper positioning thereof by reversing the direction of movement of the driver 18.

As will be appreciated by those skilled in the art, the methods shown in FIGS. 13-16 are for illustrative purposes to demonstrate the workings of the subject invention. Any configuration or method consistent with the subject invention can be utilized. For example, the stent may be balloon expandable with an expansion balloon being utilized.

In addition, as will be further appreciated by those skilled in the art, the invention can be practiced with non-limiting other variations. For example, a fail-safe relief mechanism can be provided. By way of non-limiting example, a spring tensioner can be provided to act on the driver 18 such that with the catheter 16 being stuck in the bodily passageway 68, excessive torque will cause the spring tensioner to decouple the driver 18, rather than allow for failure of one or more of the teeth 36. The exterior knobs 20 can also be provided as levers or with other shapes to generate torque or other force of movement.

1. A delivery assembly comprising:
a catheter having a lumen extending therethrough;
a housing having an aperture, said catheter extending through said aperture; and,
a driver movably coupled to said housing such that said driver can selectively move in a first direction relative to said housing, and in a second direction, different from said first direction, relative to said housing, wherein said driver is disposed to engage said catheter such that movement of said driver in said first direction causes a distal end of said catheter to move distally, and that movement of said driver in said second direction causes said distal end of said catheter to move proximally.

2. An assembly as in claim 1, wherein said driver is wheel-shaped.

3. An assembly as in claim 2, wherein said driver tangentially engages said catheter.

4. An assembly as in claim 1, wherein said first direction is a clockwise direction and said second direction is a counter-clockwise direction.

5. An assembly as in claim 1, wherein said driver frictionally engages said catheter.

6. An assembly as in claim 5, wherein said driver includes a textured or knurled surface for engaging said catheter.

7. An assembly as in claim 1, wherein said driver and said catheter are at least partially formed with shape-mating configurations.

8. An assembly as in claim 7, wherein said driver is gear-shaped with radially-spaced apart teeth, and said catheter includes corrugations, said teeth of said driver formed to mesh with said corrugations.

9. An assembly as in claim 7, wherein said catheter includes first and second portions, said first portion being generally cylindrical, said second portion including corrugations.

10. An assembly as in claim 9, wherein said first and second portions are formed of different materials.

11. An assembly as in claim 1, further comprising an idler wheel spaced from said driver, said catheter extending between said driver and said idler.

12. An assembly as in claim 1, wherein said housing includes a handle portion formed to be grippingly engaged.

13. An assembly as in claim 1, wherein said housing includes a rear port.

14. An assembly as in claim 13, further comprising a guidewire extending through said rear port and at least partially through said lumen of said catheter.

15. An assembly as in claim 14, further comprising a pusher disposed about said guidewire within said lumen.

16. An assembly as in claim 15, further comprising a stent disposed between said pusher and said catheter.

17. An assembly as in claim 1, further comprising a pusher disposed within said lumen.

18. An assembly as in claim 17, further comprising a stent disposed between said pusher and said catheter.

19. An assembly as in claim 1, wherein said driver is wholly disposed within said housing.

20. An assembly as in claim 19, further comprising at least one knob located externally of said housing, said knob being coupled to said driver such that movement of said knob results in corresponding movement of said driver.

21. An assembly as in claim 1, further comprising means for releasably locking said driver at predetermined positions relative to said housing.

22. An assembly as in claim 1, further comprising a stent disposed in said lumen, said stent being distensible to a diameter greater than said lumen.

23. A delivery assembly comprising:
a catheter having a lumen extending therethrough;
a housing having an aperture, said catheter extending through said aperture; and
driver means coupled to said housing for selectively moving a distal end of said catheter distally and proximally.
